(19) United States
(12) Patent Application Publication
(10) Pub. No.: US 2012/0082707 A1
(43) Pub. Date: Apr. 5, 2012

(54) ANTIPERSPIRANT PRODUCTS HAVING ANTIBACTERIAL PROTECTION AND PROCESSES FOR MAKING THE SAME

(75) Inventors: Travis T. Yarlagadda, Phoenix, AZ (US); Thomas Doering, Scottsdale, AZ (US)

(73) Assignee: The Dial Corporation, Scottsdale, AZ (US)

(21) Appl. No.: 12/977,315

(22) Filed: Dec. 23, 2010

Related U.S. Application Data
(60) Provisional application No. 61/388,547, filed on Sep. 30, 2010.

(51) Int. Cl. A61K 8/04; A61Q 15/00; B65B 1/04; A61K 8/92; A61K 8/02; B65D 85/84; A61K 8/29; A61K 8/58

(52) U.S. Cl. 424/401; 424/65; 424/43; 424/47; 206/524.1; 141/1

(57) ABSTRACT

A personal care product comprises an antiperspirant product that comprises an active antiperspirant compound, and titanium dioxide particles that comprise anhydrous silver compound selected from the group consisting of anhydrous silver chloride, anhydrous silver lactate, anhydrous silver citrate, and mixtures thereof.

### CROSS-REFERENCE TO RELATED APPLICATION

This application claims priority to and the benefit of U.S. Provisional Patent Application No. 61/388,547 filed Sep. 30, 2010 entitled “Anhydrous Antiperspirant Compositions Including Silver Chloride and Titanium Dioxide”, wherein such provisional application is hereby incorporated in its entirety, by reference.

### FIELD OF THE INVENTION

The present invention generally relates to personal care antiperspirant compositions, products, and processes for making the same, and more particularly relates to antiperspirant compositions that exhibit antiperspirant efficacy and that provide antibacterial protection against odors caused by bacteria, antiperspirant products comprising such antiperspirant compositions, and processes for making such antiperspirant compositions and products.

### BACKGROUND OF THE INVENTION

Antiperspirant and deodorant compositions are well known personal care products used to prevent or eliminate perspiration and body odor caused by perspiration. The compositions come in a variety of forms and may be formulated, for example, into aerosols, pumps, sprays, liquids, roll-ons, lotions, creams, sticks, and soft solids, etc.

There are various types of antiperspirant compositions that are desirable by a large majority of the population because of their ease of application and the presence of active antiperspirant compounds, e.g. antiperspirant salts, that prevent or block the secretion of perspiration and its accompanying odors. In one type, an antiperspirant salt is suspended in an anhydrous vehicle often including a solid water-insoluble wax. In a second type, an antiperspirant salt is dissolved in a liquid vehicle such as propylene glycol and gelled with a gelling agent such as dibenzylidene sorbitol. A third type includes an emulsion of an aqueous phase containing the antiperspirant salt and an oil phase containing, for example, a volatile silicone, fragrances, gellants, and other additives. In a fourth type for aerosol antiperspirant products, an antiperspirant salt is suspended in an anhydrous liquid vehicle.

Both stick and aerosol personal care antiperspirant products include an antiperspirant composition within a container. During use of the stick antiperspirant product, the top of the container is removed and the application surface of the composition is contacted with the skin, such as the underarm, by swiping or rubbing the stick across the skin. Sometimes the product also includes an undercap, or factory seal, covering the application surface that is removed prior to first use. The container generally also includes some mechanism for moving the composition upwards through the container to continue to provide an exposed application surface. During use of the aerosol antiperspirant product, the top of the container is removed and an aerosol spray valve is actuated to release the antiperspirant composition from the container onto the skin, such as the underarm.

Commercial markets for antiperspirant and deodorant products are highly competitive, with consumers wanting products with increased antiperspirant efficacy and that counteract the unwanted odors caused by bacteria, e.g., bacterial flora, present in perspiration. Current solid wax and aerosol antiperspirant products sometimes use aqueous silver compounds, such as, for example, aqueous silver lactate, aqueous silver chloride, and aqueous silver citrate, as an antibacterial agent to counteract the bacteria. Unfortunately, the water from the aqueous silver ion solution can cause aerosol antiperspirant actives to agglomerate and clog the aerosol spray valve. With regard to solid wax antiperspirant actives, the water again can cause the antiperspirant product to agglomerate resulting in a rough antiperspirant application surface. Also, the water can cause the active antiperspirant compound to lose activity thereby reducing the product's antiperspirant efficacy.

Accordingly, it is desirable to provide antiperspirant products that exhibit strong antiperspirant efficacy and that counteract the unwanted odors caused by bacteria in perspiration without agglomerating the antiperspirant actives. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Antiperspirant products and processes for manufacturing antiperspirant products are provided herein. In accordance with an exemplary embodiment, a personal care product comprises an antiperspirant product comprising an active antiperspirant compound, and titanium dioxide particles comprising anhydrous silver compound selected from the group consisting of anhydrous silver chloride, anhydrous silver lactate, anhydrous silver citrate, and mixtures thereof.

In accordance with another exemplary embodiment, an antiperspirant product is provided. The antiperspirant product comprises anhydrous silver compound that is adsorbed onto surfaces of titanium dioxide particles that are dispersed throughout at least a portion of the antiperspirant product, absorbed into the titanium dioxide particles or a combination thereof. The antiperspirant product is a solid wax formulation or an anhydrous aerosol formulation. The anhydrous silver compound is selected from the group consisting of anhydrous silver chloride, anhydrous silver lactate, anhydrous silver citrate, and mixtures thereof.

In accordance with another exemplary embodiment, a process for making an antiperspirant product is provided. The process comprises the steps of combining an active antiperspirant compound and anhydrous silver compound that is adsorbed onto surfaces of titanium dioxide particles, absorbed into the titanium dioxide particles or a combination thereof to form at least a portion of the antiperspirant product. The anhydrous silver compound is selected from the group consisting of anhydrous silver chloride, anhydrous silver lactate, anhydrous silver citrate, and mixtures thereof. The at least the portion of the antiperspirant product is deposited into a container.

### BRIEF DESCRIPTION OF THE DRAWINGS

The present invention will hereinafter be described in conjunction with the following drawing figures, wherein like numerals denote like elements, and wherein:

FIG. 1 is a perspective view of a personal care antiperspirant product in accordance with an exemplary embodiment;

FIG. 2 is a plan view of the antiperspirant product of FIG. 1;

FIG. 3 is an exploded perspective view of the antiperspirant product of FIG. 1 in accordance with an exemplary embodiment;

FIG. 4 is a perspective view of a personal care antiperspirant product in accordance with an exemplary embodiment;

FIG. 5 is a plan view of the antiperspirant product of FIG. 4;

FIG. 6 is a side cutaway view of a personal care antiperspirant product in accordance with another exemplary embodiment; and

FIG. 7 is a flowchart of an example of a process for making an antiperspirant product in accordance with the present invention.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The various embodiments contemplated herein relate to personal care antiperspirant products that exhibit antiperspirant efficacy and that provide antibacterial protection from odor-causing bacteria in perspiration. In an exemplary embodiment, the antiperspirant product comprises an active antiperspirant compound and anhydrous silver compound that is adsorbed onto and/or absorbed into titanium dioxide particles. The anhydrous silver compound can be anhydrous silver chloride, anhydrous silver lactate, anhydrous silver citrate, or mixtures thereof, and in a most preferred embodiment, the anhydrous silver compound is anhydrous silver chloride. The inventors have found that when the titanium dioxide particles are exposed to perspiration, silver ions diffuse from the titanium dioxide particles to create a silver ion equilibrium within the surrounding perspiration environment. The silver ions interact destructively with the bacteria and other microbial cells present in the perspiration and are withdrawn from the equilibrium. Further silver ions diffuse from the titanium dioxide particles to replenish and/or maintain the equilibrium. Thus, unwanted odors caused by the bacteria and other microbial cells are counteracted by the destructive interaction with the silver ions. Moreover, the source of the silver ions is from a non-aqueous form of silver salt on or in titanium dioxide particles. Thus, the antiperspirant composition can provide antibacterial protection without the use of an aqueous silver ion solution. This preferably provides an antiperspirant product with antibacterial protection in either a solid wax or aerosol form without the problems associated with agglomeration of the product and loss of activity of the active antiperspirant compound that would otherwise be caused by the presence of water.

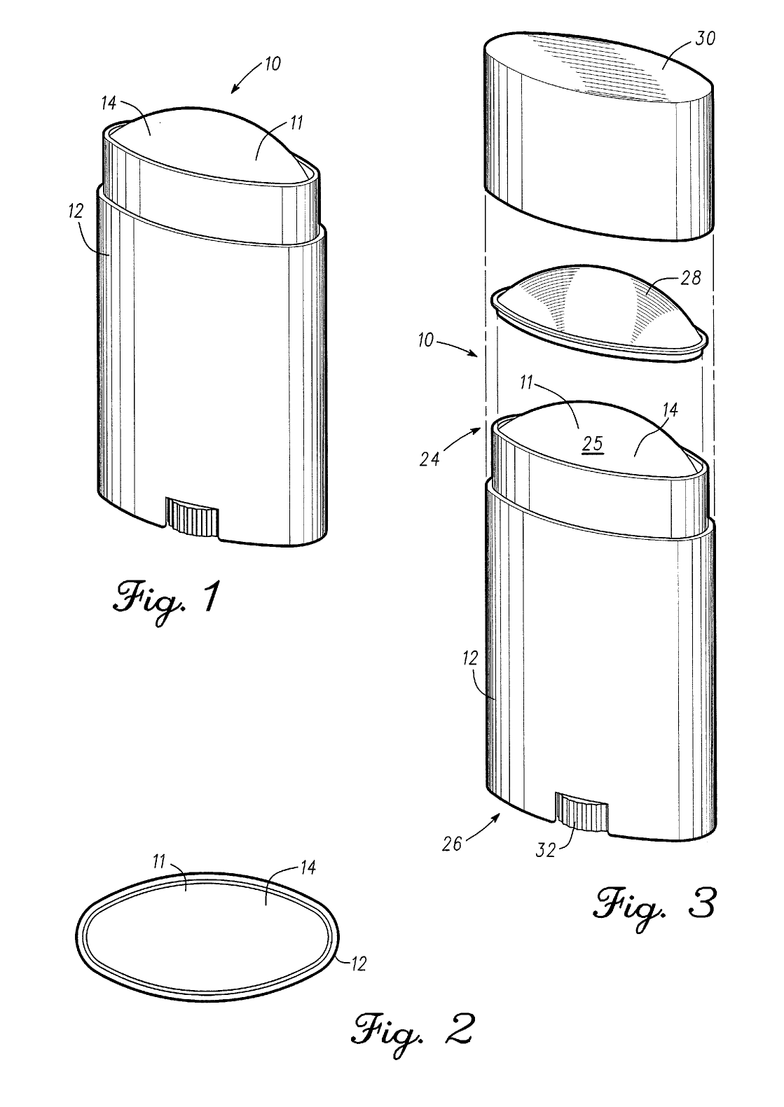

Referring to FIGS. 1 and 2, a personal care product 10 in accordance with an exemplary embodiment comprises an antiperspirant product 11. The antiperspirant product 11 is a solid wax formulation, which is preferably substantially free of water. However, other suitable antiperspirant formulations may be used, such as, for example, an antiperspirant aerosol formulation as depicted in FIG. 6. The antiperspirant product 11 has an application surface 14 that is substantially dome-shaped and that is configured to be applied to skin, such as, for example, an underarm. The personal care product 10 may also comprise a container or dispenser 12 for dispensing the antiperspirant product 11 to the skin.

In an exemplary embodiment, the antiperspirant product 11 contains anhydrous silver compound that is adsorbed onto and/or absorbed into titanium dioxide particles that are dispersed throughout the antiperspirant product 11. The inventors have found that by having anhydrous silver compound on or in the titanium dioxide particles, when the user perspires, a diffusion of silver ions from the titanium dioxide particles occurs into the hydrated environment to establish a concentration gradient. The silver ions effectively act as an antimicrobial agent that destructively interacts with odor-causing bacteria and other microbial cells. As the silver ions in the hydrated environment are consumed, more silver ions are released from the titanium dioxide particles to reestablish or maintain the concentration gradient. In one example, the titanium dioxide particles including anhydrous silver chloride on or in the titanium dioxide particles are available as JM Acticare P™, which is manufactured by Clariant in Muttenz, Switzerland.

In an exemplary embodiment, the antiperspirant product 11 has a ratio of from about 1.5:98.5 to about 20:80 of the weight of the anhydrous silver compound to the weight of the titanium dioxide particles. Preferably, the antiperspirant product 11 comprises the anhydrous silver compound together with the titanium dioxide particles in an amount of from about 0.001 to about 1 weight percent (wt. %), and more preferably, in an amount of from about 0.01 to about 0.1 wt. % of the entire antiperspirant product 11. In one exemplary embodiment, the antiperspirant product 11 comprises anhydrous silver compound in an amount of from about 0.15 to about 150 parts per million (ppm), and preferably, in an amount of from about 1.5 to about 15 ppm.

The antiperspirant product 11 may comprise an anhydrous, hydrophobic vehicle, which includes a volatile silicone and/or high melting point component, and an active antiperspirant compound suspended in the anhydrous, hydrophobic vehicle. The active antiperspirant compound is preferably in a perspiration-reducing effective amount.

The active antiperspirant compounds contain at least one active ingredient, typically metal salts, that are thought to reduce perspiration by diffusing through the sweat ducts of apocrine glands (sweat glands responsible for body odor) and hydrolyzing in the sweat ducts, where they combine with proteins to form an amorphous metal hydroxide agglomerate, plugging the sweat ducts so perspiration cannot diffuse to the skin surface. Some active antiperspirant compounds that may be used include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₙCl_{b}x(H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐx(H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zironyl hyroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the antiperspirant compound is aluminum zirconium trichlorohydrex. In another embodiment, the antiperspirant product 11 comprises an active antiperspirant compound present in the amount of 0 to about 25 wt. % (USP). As used herein, weight percent (USP) or wt. % (USP) of an antiperspirant salt is calculated as anhydrous weight percent in accordance with the U.S.P. method, as is known in the art. This calculation excludes any bound water and glycerin.

The high melting point components may include any material suitable for use in an antiperspirant stick that melts at a temperature of about 70° C. or higher. Typical of such materials are the high melting point waxes. These include beeswax, spermaceti, carnauba, bayberry, candelilla, montan, ozokerite, ceresin, paraffin waxes, semi-microcrystalline and microcrystalline waxes, hydrogenated jojoba oil, and hydrogenated castor oil (castor wax). The preferred wax is hydrogenated castor oil. Other suitable high melting point components include various types of high melting point gelling agents such as polyethylene-vinyl acetate copolymers, polyethylene homopolymers, 12-hydroxystearic acid, and substituted and unsubstituted dibenzylidene alditols. Typically, the high melting point components comprise about 1 to about 25 wt. %, preferably about 2 to about 15 wt. %, of the composition. Volatile silicones include cyclomethicones and dimethicones, discussed above.

Other components may include, for example, non-volatile silicones, polyhydric alcohols having 3-6 carbon atoms and 2-6 hydroxy groups, fatty alcohols having from 12 to 24 carbon atoms, fatty alcohol esters, fatty acid esters, fatty amides, non-volatile paraffinic hydrocarbons, polyethylene glycols, polypropylene glycols, polyethylene and/or polypropylene glycol ethers of C₄-C₂₀ alcohols, polyethylene and/or polypropylene glycol esters of fatty acids, and mixtures thereof. The term “fatty” is intended to include hydrocarbon chains of about 8 to 30 carbon atoms, preferably about 12 to 18 carbon atoms.

Non-volatile silicones include polyalkylsiloxanes, polyalkylaryl siloxanes, and polyethersiloxanes with viscosities of about 5 to about 100,000 centistokes at 25° C., polymethylphenylsiloxanes with viscosities of about 15 to about 65 centistokes, and polyoxyalkylene ether dimethylsiloxane copolymers with viscosities of about 1200 to about 1500 centistokes.

Useful polyhydric alcohols include propylene glycol, butylenes glycol, dipropylene glycol and hexylene glycol. Fatty alcohols include stearyl alcohol, cetyl alcohol, myristyl alcohol, oleyl alcohol, and lauryl alcohol. Fatty alcohol esters include myristyl lactate, C₁₂₋₁₅ alcohols benzoate, cetyl acetate, and myristyl octanoate. Fatty acid esters include isopropyl palmitate, myristyl myristate, and glyceryl monostearate. Fatty amides include stearamide MEA, stearamide MEA-stearate, lauramide DEA, and myristamide MIPA.

Non-volatile paraffinic hydrocarbons include mineral oils and branched chain hydrocarbons with about 16 to 68, preferably about 20 to 40, carbon atoms. Suitable polyethylene glycols and polypropylene glycols will typically have molecular weights of about 500 to 6000, such as PEG-10, PEG-40, PEG-150 and PPG-20, often added as rheology modifiers to alter product appearance or sensory attributes.

Polyethylene and/or polypropylene glycol ethers or C₄-C₂₀ alcohols include PPG-10 butanediol, PPG-14 butyl ether, PPG-5-buteth-7, PPG-3-isostearth-9, PPG-3-myreth-3, oleth-10, and steareth-20. Polyethylene and/or polypropylene glycol esters of fatty acids include PEG-8 distearate, PEG-10 dioleate, and PPG-26 oleate. These are generally added to give emollient properties.

The antiperspirant product 11 contemplated herein also may comprise additives, such as those used in conventional antiperspirants. For example, in addition to, or instead of, antiperspirant efficacy, the antiperspirant product 11 may comprise additives that cause the antiperspirant product 11 to exhibit long-lasting fragrance, odor protection, and/or another desired purpose and/or function. These additives include, but are not limited to, fragrances, including encapsulated fragrances, dyes, pigments, preservatives, antioxidants, moisturizers, and the like. These optional ingredients can be included in the antiperspirant product 11 in an amount of 0 to about 20 wt. %.

The above list of materials is by way of example only and is not intended to be a comprehensive list of all potential components of the antiperspirant products contemplated herein. Other high and low melting waxes, volatile and non-volatile compounds and other suitable components are readily identifiable to those skilled in the art. Of course, other ingredients such as colloidal silica, fumed silica, particulate polyolefins, talcum materials, colorants and preservatives may also be included as desired. For example, the composition may include up to about 10% fragrance or about 2% colorant by weight.

As noted above, in addition to, or instead of, an active antiperspirant compound, the antiperspirant product 11 may comprise a component or components that cause it to exhibit or impart a desired function or purpose in addition to, or instead of, antiperspirant efficacy. For example, the antiperspirant product 11 may comprise deodorant active ingredients. A suitable deodorant active ingredient is any agent that inhibits, suppresses, masks or neutralizes malodor. These may include (1) antimicrobial or bactericidal agents that kill the bacteria responsible for malodor production, (2) agents that inhibit or suppress or interfere with the bacterial enzymatic pathway that produces malodor, and (3) agents that mask or absorb or neutralize malodor, “Fragrances” as used herein are not considered deodorant active ingredients. Examples of deodorant active ingredients include triclosan, triclocarban, usnic acid salts, zinc phenolsulfonate, b-chloro-D-alanine, D-cycloserine, animooxyacetic acid, cyclodextrine, and sodium bicarbonate. Alternatively, or in addition, the antiperspirant product 11 may comprise fragrances, for example, in an amount that imparts a long-lasting fragrance to the antiperspirant product 11.

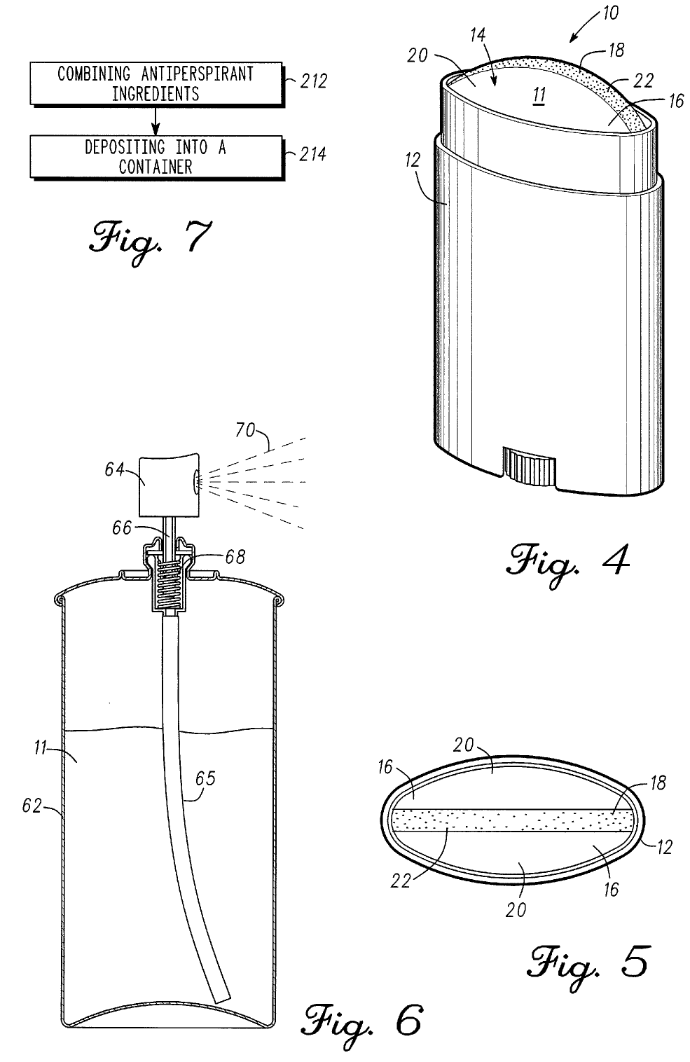

Referring to FIGS. 4 and 5, a personal care product 10 in accordance with an exemplary embodiment comprises an antiperspirant product 11 comprising a first portion 16 and a second portion 18. The term “portion,” as used herein, includes the section or sections of the antiperspirant product having the same composition; for example, two sections having the same composition but separated by a third section (for example, a central stripe) having a different composition constitute a single “portion.” The first portion 16 may have a color different from that of second portion 18 or the portions may be of the same color. As shown, the first and second portions 16 and 18 are visibly and/or macroscopically separated in non-mutual spatial relationship with each other. The antiperspirant product 11 has an application surface 14 that is substantially dome-shaped and is configured to be applied to skin, such as for example, an underarm. The personal care product 10 also may comprise a container or dispenser 12 for dispensing first portion and second portion 16 and 18 to the skin.

In one exemplary embodiment, the first portion 16 is an outer portion and the second portion 18 is an inner portion and the application surface 14 comprises a surface 20 of first portion 16 that is bisected by an adjacent surface 22 of the second portion 18. The first portion 16 and the second portion 18 are preferably solid wax formulations where at least the second portion 18 is substantially free of water. The first portion 16 is positioned on outside regions of the antiperspirant product 11 while the second portion 18 is positioned between the regions of the first portion 16. This configuration is a preferred configuration because the first portion 16 has a melting point that is higher than the melting point of the second portion 18. During manufacture, the second portion 18 is formed after the first portion 16 and thus, will not melt the already prepared first portion 16. However, it will be appreciated that the invention is not limited to the configuration of the first portion 16 and the second portion 18 illustrated in FIGS. 4 and 5. Rather, it will be appreciated that the second portion 18 can be the outer portion and the first portion 16 can be the inner portion.

It will be appreciated that the first portion 16 and the second portion 18 may also have other configurations. For example, the second portion 18 may be completely surrounded by the first portion 16 or vice versa. Alternatively, rather than forming one strip bisecting the first portion 16, the second portion 18 may form two or more strips. The antiperspirant product 11 may also comprise a third portion and other additional portions that do not comprise the compositions of the first portion 16 and the second portion 18. The first portion 16 and the second portion 18 may take any other configuration suitable for applying the portions to skin. The surface 20 of first portion 16 and the surface 22 of the second portion 18 each comprises at least 15%, and preferably at least 25%, of the application surface 14. Each surface 20 and 22 may even comprise, for example, at least 40% of the application surface 14.

In an exemplary embodiment, the first and second portions 16 and 18 are made of formulations having different compositions from each other. Preferably, the second portion 18 contains the anhydrous silver compound on or in the titanium dioxide particles as described in the foregoing paragraphs. Alternatively, the first portion 16 may contain the anhydrous silver compound on or in the titanium dioxide particles. In one example, the second portion 18 comprises the anhydrous silver compound together with the titanium dioxide particles in an amount of from about 0.01 to about 2 wt. % of the entire antiperspirant product 11. In another example, the second portion 18 comprises the anhydrous silver compound in an amount of from about 1.5 to about 300 ppm. Additionally, the first and second portions 16 and 18 may each contain one or more ingredients as discussed above in reference to the solid wax antiperspirant product 11 depicted in FIGS. 1 and 2, such as, for example, active antiperspirant compounds, structurants, high melting point components, non-volatile silicones, polyhydric alcohols, polyethylene and/or polypropylene glycol ethers, additives, fragrances, etc.

Referring to FIG. 6, a personal care product 10 in accordance with another exemplary embodiment is provided. The personal care product 10 comprises an antiperspirant product 11 having an anhydrous aerosol formulation, which is preferably substantially free of water and is stored in a container 62. The aerosol formulation comprises the anhydrous silver compound on or in the titanium dioxide particles and includes other various ingredients as discussed in the foregoing paragraphs, such as, for example, an active antiperspirant compound, talc, a volatile silicone, and one or more fragrances, and further includes a gaseous propellant, such as, one or more types of light hydrocarbons and/or fluorocarbons. The antiperspirant product 11 is contained in the container 62 under pressure from the gaseous propellant. When the plunger cap 64 is depressed, the valve stem 66 is actuated against a spring 68 to an open position allowing the liquid antiperspirant product 11 to be forced, via a gaseous propellant, through a tube 65, the valve stem 66 and finally out of a small hole in the plunger cap 64 as an aerosol or mist 70. Other suitable aerosol valve-container arrangements known to those skilled in the art may also be used with the various embodiments of the present invention.

In accordance with at least one exemplary embodiment, a process for making the antiperspirant product illustrated in FIGS. 1-2, and 4-6 is shown in FIG. 7. With reference to FIG. 7, the process generally denoted at 210 comprises combining an active antiperspirant compound and anhydrous silver compound that is absorbed onto surfaces of titanium dioxide particles, absorbed into the titanium dioxide particles or a combination thereof to form at least a portion of the antiperspirant product (step 212). The mixture of the active antiperspirant compound and the titanium dioxide particles with the anhydrous silver compound is deposited into a mold/container (step 214).

In an exemplary embodiment, the antiperspirant 11 depicted in FIGS. 1-2 is made by mixing together the active antiperspirant compound and the anhydrous silver compound on the titanium dioxide particles (step 212). In one example, the active antiperspirant compound and the anhydrous silver compound on the titanium dioxide particles are mixed together with other antiperspirant ingredients, such as, for example, a structurant, hot melt waxes, silicones, polyhydric alcohols, paraffinic hydrocarbons, glycols, and/or etc. to form the antiperspirant product 11.

The antiperspirant product 11 is formed by depositing the antiperspirant product 11 in molten form into a mold (step 214) and solidifying the antiperspirant product 11. The molten temperature of the antiperspirant product 11 is generally in the range of from about 65 to about 85° C., and the antiperspirant product is cooled below about 40° C., to produce the antiperspirant product 11 in a non-molten form. A container 12 may be used as the mold for the antiperspirant product to form the antiperspirant product 11 illustrated in FIG. 3. It will be appreciated, however, that the invention is not limited to use of the container 12 as a mold and that any satisfactory mold may be used for manufacturing the antiperspirant product.

Referring to FIG. 3, in one embodiment, the container 12 has an application end 24 and an opposite end 26. The container 12 also contains a factory seal 28, which is positioned over the application surface 14 of antiperspirant product 11 to protect it during shipment and to render it tamper-proof prior to purchase, and a cover 30. The factory seal 28 is removed by the user, and the cover is used during storage of the product between uses. As the product is exhausted, it is advanced from the container 12 by the user using advancement device 32, e.g., a screw mechanism as shown, at opposite end 26 of container 12.

As discussed in U.S. Pat. No. 5,753,212 filed Sep. 16, 1996, which is incorporated herein by reference in its entirety, the antiperspirant product 11 may be molded into the container 12 by first sealing the application end 24 of the container 12 with the factory seal 28 and introducing a predetermined quantity of molten antiperspirant product 11 through the open opposite end 26. The antiperspirant product 11 is then cooled to its non-molten form for example, by passing the filled container through a forced air tunnel operating at between about 10 to about 25° C. Alternatively, the antiperspirant product 11 may be cooled to its non-molten form by exposure to room temperature ambient conditions. The finished product is completed by sealing the open opposite end 26 with a package base (not shown) that includes the advancement device 32. Other suitable methods for molding and/or forming the antiperspirant product 11 known to those skilled in the art may also be used.

In an exemplary embodiment and with reference to FIG. 7, the antiperspirant 11 depicted in FIGS. 4-5 is made by mixing antiperspirant ingredients together to form the first portion 16, and further, by mixing a second active antiperspirant compound and the anhydrous silver compound on or in the titanium dioxide particles together to form the second portion 18 (step 212). In one example, a first active antiperspirant compound is mixed together with other antiperspirant ingredients, such as, for example, a structurant, hot melt waxes, silicones, polyhydric alcohols, paraffinic hydrocarbons, glycols, and/or etc. to form the first portion 16. Additionally, the second active antiperspirant compound and the anhydrous silver compound on or in the titanium dioxide particles are mixed together with other antiperspirant ingredients, such as, for example, a structurant, hot melt waxes, silicones, polyhydric alcohols, paraffinic hydrocarbons, glycols, and/or etc. to form the second portion 18. In another example, the first and second active antiperspirant compound are the same active antiperspirant compound and are initially mixed with other antiperspirant ingredients, such as, for example, a silicone fluid and silica to form a premix. The premix is then used to form two separate and distinct mixes with additional antiperspirant ingredients to form the corresponding first and second portions 16 and 18.

In an exemplary embodiment, the antiperspirant product 11 depicted in FIGS. 4-5 is molded directly into a mold (step 214), which is preferably the container 12 (see also FIG. 3 for the container assembly), by sequentially depositing the first and second portions 16 and 18 into the mold. In one example, as discussed in U.S. Pat. No. 6,723,269 filed Feb. 15, 2001, which is incorporated herein by reference in its entirety, the antiperspirant product 11 is molded into the container 12 by first sealing the application end 24 of the container 12 with the factory seal 28. A molding surface is inserted into the container 12 through the open opposite end 26 to define a molding cavity for the first portion 16. A predetermined quantity of the first portion 16 in molten form is delivered to the open opposite end 26 through a first delivering nozzle to form the first portion 16. The first portion 16 is allowed to partially solidify. After the first portion 16 has sufficiently solidified to prevent intermingling of the first and second portions 16 and 18, a predetermined quantity of the second portion 18 in molten form is delivered to the open opposite end 26 through a second delivery nozzle to form the second portion 18. Preferably, the first portion 16 has a slightly higher melting point than the second portion 18. The molding surface may be removed from the container 12 either prior to delivery of the molten second portion 18 or during delivery of the molten second portion 18. The antiperspirant product 11 is then cooled and the finished product is completely sealed by sealing the open opposite end 26 with a package base that includes the advancement device 32. Other suitable methods for molding and/or forming the antiperspirant product 11 known to those skilled in the art may also be used.

In another exemplary embodiment and with reference to FIG. 7, the antiperspirant product 11 depicted in FIG. 6 is made by mixing together the active antiperspirant compound and the anhydrous silver compound on or in the titanium dioxide particles (step 212). In one example, the active antiperspirant compound and the anhydrous silver compound on the titanium dioxide particles are mixed together with other antiperspirant ingredients, such as, for example, talc, one or more fragrances, and a volatile silicone. The mixture is then deposited into the container 62 (step 214), which is then charged with a gaseous propellant, such as, one or more types of light hydrocarbons and/or fluorocarbons.

The following are examples of the antiperspirant product in accordance with the present invention. The examples are provided for illustration purposes only and are not meant to limit the various embodiments of the antiperspirant product in any way. All materials are set forth in weight percent.

### EXAMPLES

### Antiperspirant Product Composition

### Example 1

### Antiperspirant Product—Solid Wax Formulation

| Ingredient | Wt. % |
|---|---|
| Cyclopentasiloxane | 25-45 |
| Stearyl Alcohol | 15-22 |
| Aluminum Zironium | 15-25 |
| Trichlorohydrex GLY | |
| PPG-14 Butyl Ether | 4-15 |
| Hydrogenated Castor Oil | 2-8 |
| Fragrance | 0.1-4 |
| Myristal Myristate | 0.1-4 |
| Silica | 0.1-4 |
| Silica Dimethyl Silylate | 0.1-4 |
| JM Acticare P ™ | 0.001-1 |
| Total | 100.0 |

### Example 2

### Antiperspirant Product—Solid Wax Formulation with Outer and Inner Portions

| Active Premix - Ingredient | Wt. % |
|---|---|
| Aluminum Zirconium Trichlorohydrex GLY | 25-75 |
| Cyclopentasiloxane | 25-75 |
| Aerosil 300 (Silica) | 0.1-4 |
| Aerosil R 972 V (Fumed Silica) | 0.1-4 |
| Total | 100.0 |

| Outer Portion - Ingredient | Wt. % |
|---|---|
| Active Premix | 25-75 |
| Cyclopentasiloxane | 4-15 |
| Castor Oil, Hydrogenated | 2-8 |
| Myristal Myristate | 0.1-4 |
| Fluid AP, Low Odor Bulk | 4-15 |
| Stearyl Alcohol, Solid | 15-22 |
| Total | 100.0 |

| Inner Portion - Ingredient | Wt. % |
|---|---|
| Active Premix | 25-75 |
| Cyclopentasiloxane | 4-15 |
| Stearyl Alcohol, Solid | 15-22 |
| Castor Oil, Hydrogenated | 0.1-4 |
| Myristyl Myristate | 0.1-4 |
| Fluid AP, Low Odor Bulk | 4-15 |
| Aluminum Powder | 0.01-2 |
| Fragrance | 2-8 |
| SYM 388782 Triton 70% Incap 197341/PSM/60 | 0.1-4 |
| Protectate | 0.1-4 |
| JM Acticare P ™ | 0.01-2 |
| Total | 100.0 |

### Example 3

### Antiperspirant Product—Anhydrous Aerosol Formulation

| Ingredient | Wt. % |
|---|---|
| Butane | 25-45 |
| Cyclomethicone | 20-45 |
| Hydrofluorocarbon | 10-30 |
| Aluminum Chlorohydrate | 4-15 |
| Talc | 0.1-4 |
| Silica Dimethyl Silylate | 0.1-4 |
| Fragrance | 0.1-4 |
| Silica | 0.1-4 |
| JM Acticare P ™ | 0.001-1 |
| Total | 100.0 |

Accordingly, personal care antiperspirant products that exhibit antiperspirant efficacy and which provide antibacterial protection from odor-causing bacteria in perspiration have been described. The various embodiments of the antiperspirant products comprise an active antiperspirant compound and anhydrous silver compound that is adsorbed onto and/or absorbed into titanium dioxide particles. When the titanium dioxide particles are exposed to perspiration, silver ions diffuse from the titanium dioxide particles to create a silver ion equilibrium within the surrounding perspiration environment. The silver ions interact destructively with the bacteria and other microbial cells present in the perspiration and are withdrawn from the equilibrium. Further silver ions diffuse from the titanium dioxide particles to replenish and/or maintain the equilibrium. Thus, unwanted odors caused by the bacteria and other microbial cells are counteracted by the destructive interaction with the silver ions. Moreover, the source of the silver ions is from a non-aqueous form (i.e. anhydrous form) of silver salt on or in titanium dioxide particles. Thus, the antiperspirant composition can provide antibacterial protection without the use of an aqueous silver ion solution. This preferably provides an antiperspirant product with antibacterial protection in either a solid wax or aerosol form without the problems associated with agglomeration of the product and loss of activity of the active antiperspirant compound that would otherwise be caused by the presence of water.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A personal care product comprising:
an antiperspirant product comprising:
an active antiperspirant compound; and
titanium dioxide particles comprising anhydrous silver compound selected from the group consisting of anhydrous silver chloride, anhydrous silver lactate, anhydrous silver citrate, and mixtures thereof.

2. The personal care product according to claim 1, wherein the antiperspirant product has a weight ratio of from about 1.5:98.5 to about 20:80 of the anhydrous silver compound to the titanium dioxide particles.

3. The personal care product according to claim 1, wherein the antiperspirant product comprises the anhydrous silver compound with the titanium dioxide particles in an amount of from about 0.001 to about 1 wt. % of the antiperspirant product.

4. The personal care product according to claim 1, wherein the antiperspirant product comprises the anhydrous silver compound with the titanium dioxide particles in an amount of from about 0.01 to about 0.1 wt. % of the antiperspirant product.

5. The personal care product according to claim 1, wherein the antiperspirant product comprises the anhydrous silver compound in an amount of from about 0.15 to about 150 ppm.

6. The personal care product according to claim 1, wherein the antiperspirant product comprises the anhydrous silver compound in an amount of from about 1.5 to about 15 ppm.

7. The personal care product according to claim 1, further comprising a container housing the antiperspirant product.

8. The personal care product according to claim 1, wherein the antiperspirant product is a solid wax formulation.

9. The personal care product according to claim 1, wherein the antiperspirant product has a first portion and a second portion that is macroscopically separated from the first portion, the second portion having a composition different from the first portion and comprising the titanium dioxide particles that comprise the anhydrous silver compound.

10. The personal care product according to claim 9, wherein the second portion comprises the anhydrous silver compound with the titanium dioxide particles in an amount of from about 0.01 to about 2 wt. % of the second portion.

11. The personal care product according to claim 9, wherein the second portion comprises the anhydrous silver compound in an amount of from about 1.5 to about 300 ppm.

12. The personal care product according to claim 9, wherein the first portion comprises a first region and a second region and the second portion is positioned between the first and second regions.

13. The personal care product according to claim 1, wherein the antiperspirant product is an anhydrous aerosol formulation.

14. An antiperspirant product comprising anhydrous silver compound that is adsorbed onto surfaces of titanium dioxide particles that are dispersed throughout at least a portion of the antiperspirant product, absorbed into the titanium dioxide particles or a combination thereof, wherein the antiperspirant product is a solid wax formulation or an anhydrous aerosol formulation, and wherein the anhydrous silver compound is selected from the group consisting of anhydrous silver chloride, anhydrous silver lactate, anhydrous silver citrate, and mixtures thereof.

15. A process for making an antiperspirant product comprising the steps of:
combining an active antiperspirant compound and anhydrous silver compound that is adsorbed onto surfaces of titanium dioxide particles, absorbed into the titanium dioxide particles or a combination thereof to form at least a portion of the antiperspirant product, wherein the anhydrous silver compound is selected from the group consisting of anhydrous silver chloride, anhydrous silver lactate, anhydrous silver citrate, and mixtures thereof; and
depositing the at least the portion of the antiperspirant product into a container.

16. The process according to claim 15, wherein the step of combining the active antiperspirant compound and the anhydrous silver compound comprises forming the antiperspirant product having a weight ratio of from about 1.5:98.5 to about 20:80 of the anhydrous silver compound to the titanium dioxide particles.

17. The process according to claim 15, wherein the step of combining the active antiperspirant compound and the anhydrous silver compound comprises forming the antiperspirant product comprising the anhydrous silver compound with the titanium dioxide particles in an amount of from about 0.001 to about 1 wt. % of the antiperspirant product.

18. The process according to claim 15, wherein the step of combining the active antiperspirant compound and the anhydrous silver compound comprises forming the antiperspirant product comprising the anhydrous silver compound with the titanium dioxide particles in an amount of from about 0.01 to about 0.1 wt. % of the antiperspirant product.

19. The process according to claim 15, wherein the step of combining the active antiperspirant compound and the anhydrous silver compound comprises forming the antiperspirant product comprising the anhydrous silver chloride in an amount of from about 0.15 to about 150 ppm.

20. The process according to claim 15, wherein the step of combining the active antiperspirant compound and anhydrous silver compound comprises forming the antiperspirant product comprising the anhydrous silver compound in an amount of from about 1.5 to about 15 ppm.
